(19) United States
(12) Patent Application Publication
(10) Pub. No.: US 2010/0222416 A1
(43) Pub. Date: Sep. 2, 2010

(54) Diagnostic Marker and Therapeutic Target for Cancer

(76) Inventors: Kapil Maithal, Uttar Pradesh (IN); Harish Chandra, Uttar Pradesh (IN); Rama Mukherjee, Uttar Pradesh (IN)

Correspondence Address: LADAS & PARRY LLP, 26 WEST 61ST STREET, NEW YORK, NY, 10023, US

(21) Appl. No.: 12/083,366

(22) PCT Filed: Oct. 6, 2006
(86) PCT No.: PCT/IN2006/000402

§ 371 Date: Apr. 23, 2010

(30) Foreign Application Priority Data
Oct. 13, 2005 (IN) 2748-DEL-2005

(51) Int. Cl. A61K 31/7088; C07H 21/04; C12Q 1/68; C12Q 1/02; A61K 48/00; A61P 35/04

(52) U.S. Cl. 514/44 R; 536/23.1; 435/6; 435/29

(57) ABSTRACT

The present invention, in general, relates to use of nm23H1-B gene for diagnosing cancer susceptibility and metastatic progression in colon/colorectal cancer. In particular, the present invention relates to identifying the alteration in the expression of nm23H1-B gene on treatment with chemotherapeutic agents for diagnosing cancer susceptibility and metastatic progression in colon/colorectal cancer.

### FIELD OF THE INVENTION

The present invention relates to a method for diagnosing cancer susceptibility and metastatic progression, particularly in colon/colorectal cancer, said method comprising identifying the alteration in the expression of nm23H1-B gene. Particularly, the present invention relates to a method of diagnosing cancer susceptibility and metastatic progression on treatment with different chemotherapeutic agents particularly 5-FU in colon cancer, said method comprising identifying the alteration in the expression of nm23H1-B gene. More particularly, the present invention relates to the use of the nm23H1-B gene as a therapeutic agent, for the treatment of cancer. More particularly, the present invention relates to a sensitivity diagnostic marker and a therapeutic target for various cancers, more particularly colon/colorectal cancer, wherein the said marker is nm23H1B gene. The present invention provides the predominant localization of nm23H1-A gene and nm23H1-B gene in the cell.

### BACKGROUND OF THE INVENTION

Cancer is the general name for over 100 medical conditions involving uncontrolled and dangerous cell growth. Cancer of skin, bladder, breast, colon, lung and pancreas are diagnosed with the greatest frequency and are therefore, also referred as common cancer. Colon/colorectal carcinoma are most common forms of cancer. Despite medical advances in diagnosis and treatment, unfortunately, the overwhelming majority of patients with colon/colorectal carcinoma would die of metastatic disease [Budenholzer B. Screening for colon/colorectal cancer. CMAJ 2001; 164: 965-966]

Studies from the past decade showed adjuvant chemotherapy with 5-FU, results in a significant reduction in mortality, related to colon/colorectal cancer. An improvement in survival is also observed after systemic chemotherapy for metastatic colon/colorectal cancer. Unfortunately, patient responds very differently to the treatment. For some patients, the standard dose is too toxic, whereas other patients have minimal side effects. There is also substantial variability in tumor response or survival after chemotherapy. The mechanistic basis for the different reaction and/or unsuccessful treatment is rarely known. Majority of colon/colorectal carcinomas arise from a series of somatic genetic changes that involve activation of oncogenes and inactivation of tumor suppressor genes. The delineation of molecular genetic and biological changes that accompany the pathogenesis of colon/colorectal carcinoma will hopefully improve the outcome of patients in the future.

Unlike the molecular events described for the pathogenesis of primary colon/colorectal carcinomas, genes responsible for metastasis in these tumors have not been well characterized. Exploring metastasis related genes is significantly important in the prevention of tumor metastasis and prolongation of the life expectancy of patients.

Recently, many such attempts have been made in identifying such genes and their relation with other markers like Tiam1 [Liu L, Wu D, Ding Y. Tiam1 gene expression and its significance in colon carcinoma. World J Gastroenterol 2005; 11(5): 705-707], MMPs [Choi J, Choi K, Benveniste E N, Hong Y, Lee 3, Kim J, Park K. Bcl-2 promotes invasion and lung metastasis by inducing matrix metalloproteinase-2. Cancer Res. 2005; 65(13): 5554-5560] and especially nm23H1 [Hartsough M, Steeg P. The nm23/Nucleoside diphosphate kinase in human cancers. J. Bioenerg. Biomembr. 2000; 32: 301-308].

The nm23 gene also known as nucleoside diphosphate kinase was identified as a metastasis suppressor gene [Steeg P S, Bevilacqua G, Kopper L, Thorgeirsson U P, Talmadge J B, Liotta L A, Sobel M E. Evidence for a novel gene associated with low tumor metastatic potential. J. Natl. Cancer Inst. 1988; 24: 200-204]. Further, transfection studies of nm23 cDNA into various cancer cell lines resulted in the suppression of metastatic potential of motility, invasion or colonization [Leone A, Flatow U, King C R, Sandeen M A, Margulies I M, Liotta L A, Steeg P S. Reduced tumor incidence, metastatic potential and cytoplasmic responsiveness of nm23 transfected melanoma cells. Cell 1991; 65: 25-35; Leone A, Flatow U, VanHoutte K, Steeg P S. Transfection of human nm23-H1 into the human MDA-MB-435 breast carcinoma cell line: effects on tumor metastatic potential, colonization and enzymatic activity. Oncogene. 1993; 8: 2325-2333; Liu F, Zhang Y, Zhang X Y, Chen H L. Transfection of the nm23-H1 gene into human hepatocarcinoma cell line inhibits the expression of sialyl Lewis X, alpha1, 3 fucosyltransferase VII, and metastatic potential. J. Cancer Res. Clin. Oncol. 2002; 128: 189-196; Khan M H, Yasuda M, Higashino F, Haque S, Kohgo T, Nakamura M, Shindoh M. nm23-H1 suppresses invasion of oral squamous cell carcinoma-derived cell lines without modifying matrix metalloproteinase-2 and matrix metalloproteinase-9 expression. Am. J. Pathol. 2001; 158: 1785-1791; Lee H Y, Lee H. Inhibitory activity of nm23-H1 on invasion and colonization of human prostate carcinoma cells is not mediated by its NDP kinase activity. Cancer Lett. 1999; 145: 93-99; Lim S, Lee H Y, Lee H. Inhibition of colonization and cell-matrix adhesion after nm23-H1 transfection of human prostate carcinoma cells. Cancer Lett. 1998; 133: 143-149). This suggests that nm23 could function on the invasion and migration steps of the metastatic pathway.

Eight human nm23 genes have been characterized so far. The initial 4 human genes of this family, nm23H1, nm23H2, nm23H3 (or DR-nm23) and nm23H4, encode proteins that possess NDP kinase activity and are named NDP kinase A-D, respectively. Of the 8 human nm23 genes, the H1 gene is, most closely correlated with the metastatic phenotype in human breast, colon/colorectal and ovarian carcinoma. [Stahl J A, Leone A, Rosengard A M, Porter L, King C R, Steeg P S. Identification of a second human nm23 gene, nm23-H2. Cancer Res. 1991; 31: 445-449; Tannapfel A, Kockerling F, Katalinic A, Wittekind C. Expression of nm23-H1 predicts lymph node involvement in colorectal carcinoma. Dis. Colon Rectum 1995; 38: 651-654; Viel A, Dall A L, Canzonieri V, Sopracordevole F, Capozzi E, Carbone A, Visentin M C, Boiocchi M. Suppressive role of the metastasis related nm23-H1 gene in human ovarian node metastasis. Cancer Res. 1995; 55: 2645-2650; Lacombe M L, Milon L, Munier A, Mehus J G, Lambeth D O. The human Nm23/nucleoside diphosphate kinases. J. Bioenerg. Biomembr. 2000; 32: 247-258; Masse K, Dabemat S, Bourbon P M, Larou M, Amrein L, Barraud P, Perel Y, Camara M, Landry M, Lacombe M L, Daniel J Y. Characterization of the nm23-M2, nm23-M3 and nm23-M4 mouse genes: comparison with their human orthologs. Gene 2002; 296: 87-97].

The cellular mechanisms by which the nm23H1 protein modulates the metastatic phenotype are not yet known; however, several studies reported that nm23H1 inhibited the cell motility toward platelet derived growth factor (PDGF) and insulin-like growth factor (IGF) [Kantor J D, McCormick B, Steeg P S, Zetter B R. Inhibition of cell motility after nm23 transfection of human and murine tumor cells. Cancer Res. 1993; 53:1971-1973; Russel R L, Pedersen A N, Kantor J, Geisinger K, Long R, Zbieranski N, Townsend A, Shelton B, Brunner N, Kute T E. Relationship of nm23 to proteolytic factors, proloferation and motility in breast cancer tissues and cell lines. Br. J. Cancer. 1998; 78: 710-717]. The nm23H1, an anti-metastasis gene, has been reported to correlate with sensitivity to chemotherapeutic agents including cisplatin in human oesophageal squamous cell carcinoma (OSCC) although they did not find any effect of etoposide and 5-FU in OSCC [Iizuka N, Hirose K, Noma T, Hazama S, Tangoku A, Hayashi H, Abe T, Yamamoto K, Oka M. The nm23-H1 gene as a predictor of sensitivity to chemotherapeutic agents in oesophageal squamous cell carcinoma. Br. J. Cancer 1999; 81(3): 469-475]

Recently a new transcript of nm23H1 gene viz. nm23H1-B has been identified from 18-week-old human fetal brain [Ni X, Gu S, Dai J, Cheng H, Guo L, Li L, Ji C, Xie Y, Ying K, Mao Y. Isolation and characterization of a novel human NM23-H1B gene, a different transcript of NM23-H1. J. Hum. Genet. 2003; 48: 96-100]. This 987-bp cDNA encodes a protein of 177 amino acid residues. Compared with nm23H1 (or nm23H1-A), the cDNA contained an additional 25 amino acid residues at the NH2-terminal region (FIG. 1). It was mapped to chromosome 17q21.3 using bioinformatics analysis, which shows that the second exon does not exist in nm23H1 (or nm23H1-A).

The expression pattern of nm23H1-B showed that it was ubiquitously present in most of the normal tissues (15 tissues except colon) at different levels but the expression of the transcript in tumor cells was related to tumor differentiation. In poorly differentiated breast carcinoma GI-101, pancreatic adenocarcinoma GI-103, and undifferentiated ovarian carcinoma GI-101, there was no expression. In poorly differentiated lung carcinoma LX-1, lung carcinoma GI-117, the expression level was very low. The transcript band in well-differentiated colon adenocarcinoma CX-1 was significantly, higher than that in poorly differentiated colon adenocarcinoma GI-112. [Ni X, Gu S, Dai J, Cheng H, Guo L, Li L, Ji C, Xie Y, Ying K, Mao Y. Isolation and characterization of a novel human NM23-H1B gene, a different transcript of NM23-H1. J. Hum. Genet. 2003; 48: 96-100].

A recent report, has also reported similar findings in ovarian cancer with the expression of this gene being elevated in stage I and II of cancer and subsequently going down in stage III and IV [Li W, Liu Y, Jin Z J, Feng Y J, Huang L M, Chen J. Study on mRNA expression of the human novel gene NM23-H1B in ovarian tumor (Article in Chinese). Zhonghua Fu Chan Ke Za Zhi. 2006; 41(1): 48-51].

While, there are studies reporting the over expression of nm23H1-B gene as well as nm23H1-A gene [Miyazake H, Fukuda M, Ishijima Y, Takagi Y, Iimura T, Negish A, Hirayama R, Ishikawa N, Amagasa T, Kimura N. Over expression of nm23-H2/NDP kinase B in a human oral squamous cell carcinoma cell line results in reduced metastatic site, and growth factor independent proliferative activity in culture. Clinical Cancer Research 1999; 5, 4301-4307], however such studies report only the expression level of such genes in carcinoma cell lines.

However, till date there is no report of any over expression of nm23H1-B gene in various cancer cell lines along with a chemotherapeutic agent.

Such an over expression of nm23H1-B gene in combination with chemotherapeutic agents, if found would not only render the said gene to be useful as a therapeutic target and diagnostic marker in cancer therapy but also help in meeting the requirement of a long felt need for such a prognostic and diagnostic marker in the field of invention described herein.

The present invention is a step forward in this direction, which is detailed herein below.

### OBJECTS OF THE INVENTION

An object of the present invention is to provide a method for diagnosing cancer susceptibility and metastatic progression in colon/colorectal cancer based on identifying the alteration in the expression of nm23H1-B gene on treatment with chemotherapeutic agents.

Another object of the present invention is to provide data on the expression of nm23H1-B gene both at protein and gene level in tumor cells especially colon/colorectal cancer cells on treatment with various chemotherapeutic agents.

Yet another object of the present invention is to provide a sensitive prognostic and diagnostic marker and a therapeutic target for various cancers, more particularly colon/colorectal cancer, wherein the said marker is nm23H1B gene.

Yet a further object of the present invention is to provide the predominant localization of nm23H1B and nm23H1-A gene in the cell.

Another object of the present invention is to make use of a gene as a therapeutic agent, wherein the gene is the nm23H1B gene.

### SUMMARY OF THE INVENTION

The present inventors have found that nm23H1-B gene as well as the protein encoded by it is up regulated on treatment with various chemotherapeutic agents, in particular 5-FU, in comparison to nm23H1-A gene. The up regulation exhibited by nm23H1-B gene is significantly more than that exhibited by nm23H1-A gene under identical conditions which suggests that nm23H1-B gene could be used as a prognostic and diagnostic marker and a therapeutic target for various cancers. This would be evident from analysis of mRNA levels of nm23H1-B gene expression in two cancer cell lines viz. colo-205 and HT-29 on treatment with 5-FU as will be described hereinafter.

Similarly, it was found that nm23H1-B gene as well as protein encoded by it also exhibited similar up regulation of mRNA levels in HT-29 cell line on treatment with other chemotherapeutic agents such as cisplatin and camptothecin-11 as will be described hereinafter.

Such an over expression exhibited by nm23H1-B gene both at gene and protein level in tumor cells especially colon/colorectal cancer cells render its usefulness in diagnosing cancer susceptibility and metastatic progression in colon/colorectal cancer based on identifying the alteration in the expression of nm23H1-B gene on treatment with chemotherapeutic agents.

It was also found that the metastatic potential of nm23H1-B-transfected cell line was substantially attenuated, which was more than that of nm23H1A-transfected cell line, suggesting a much more important role of nm23H1B than commonly discussed nm23H1A form of nm23H1 gene as will be described hereinafter.

Besides, it was also found that the predominant localization of nm23H1B form in the cytosolic fraction, whereas the nm23H1A form is localized in the nuclear fraction as will be described hereinafter.

### BRIEF DESCRIPTION OF THE DRAWINGS

FIGS. 1A, 1B and 1C: The gene sequence (SEQ ID NO:1) and the amino acid sequence (SEQ ID NO:2) of the human nm23H1-B and sequence alignment of nm23H1-B with nm23H1-A. Numbers on the right refer to the last amino acid in each corresponding line.

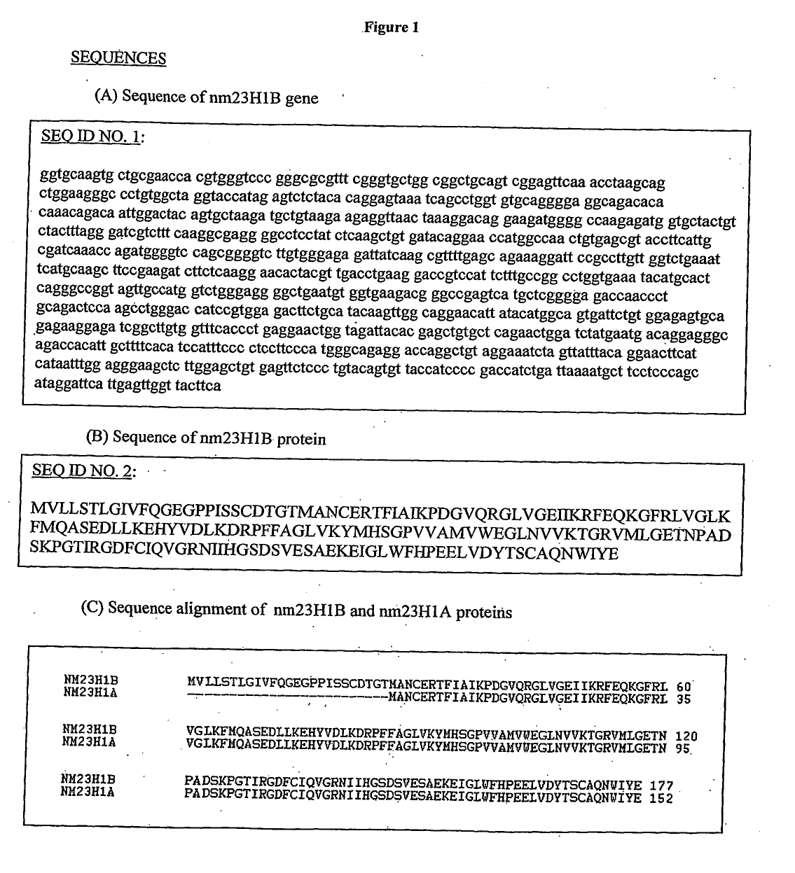

FIG. 2: Left Panel: Zoomed in region of a two-dimensional PAGE showing the over expression of nm23H1 protein on treatment with 5-FU as compared with control (cells not treated with 5FU). Right Panel: Densitometric evaluation of the intensity (volume) of the nm23H1-B spot as a percentage value of the total volume of all the spots on the gel. The analysis was done using PDQuest ver 7.2 software and is an average of six independent experiments.

FIG. 3: mRNA levels of nm23H1-A and nm23H1-B on treatment of colo-205 and HT29 cells with 5-FU as determined by RT-PCR using gene specific primers. 1% agarose gel showing differential mRNA levels of nm23H1-A and nm23H1-B for colo-205 (a) and HT29 (b) cells respectively. β-microglobulin (β₂-m) is used as a control. Lane 1: DNA marker, Lane 2&4: Untreated sample, Lane 3&5: 5-FU treated sample. Graphical representation showing the selective increase in the mRNA level of nm23H1-B for colo-205 and HT29 cells shown in (c) and (d) respectively. The mRNA level of individual genes in untreated samples is taken as one.

FIG. 4: Increase in the mRNA levels of nm23H1-B in HT29 cell line on treatment with 5-FU (a.) as a function of time, (b.) as a function of concentration of 5-FU for a 24H assay monitored on real time PCR. Glyceraldehyde-3-phosphate dehydrogenase (GAPDH) is used as a control.

FIG. 5: Cellular localization and differential expression of nm23H1-A and nm23H1-B proteins. Lane 1: 5-FU treated nuclear fraction, Lane 2: Untreated nuclear fraction, Lane 3: 5-FU treated cytosolic fraction, Lane 4: Untreated cytosolic fraction.

FIG. 6: Increase in the mRNA levels of nm23H1-A and nm23H1-B in HT29 cell line on treatment with 5-FU, CPT11, cisplatin and paclitaxel as a function of time monitored by RT-PCR and by real time PCR. β-microglobulin (β₂-m) is used as a control in RT-PCR analysis and GAPDH is used as a control in real time PCR analysis.

### DETAILED DESCRIPTION OF THE INVENTION

The present invention provides method for diagnosing cancer susceptibility and metastatic progression in colon/colorectal cancer based on identifying the alteration in the expression of nm23H1B gene on treatment with chemotherapeutic agents such as 5 FU, CPT 11, cisplatin etc.

Over expression of nm23H1-B gene as compared to nm23H1-A, both at protein and gene level in tumor cells is found in colon/colorectal cancer cells, on treatment with various chemotherapeutic agents, with about nine fold overexpression of nm23H1-B gene for 5FU, hence providing the method for diagnosing cancer susceptibility and metastatic progression in colon/colorectal cancer.

The present invention also provides that the predominant localization of nm23H1B form is in the cytosolic fraction, whereas the nm23H1A form is localized in the nuclear fraction.

The present invention also provides the use of the gene as a therapeutic agent, wherein the gene is the nm23H1 B. The invention also shows that the metastatic potential nm23H1-B-transfected cell line was much more attenuated than that of nm23H1A-transfected cell line, suggesting a much more important role of nm23H1B than commonly discussed nm23H1A form.

In view of the above, the invention provides a sensitive prognostic and diagnostic marker and a therapeutic target for various cancers, more particularly colon/colorectal cancer, wherein the said marker is nm23H1B.

The invention will be further described with respect to the following examples; however, the scope of the invention is not to be limited thereby.

### Example 1

### Two-Dimensional Polyacrylamide Gel Electrophoresis (PAGE), Image Analysis, In-Gel Digestion and MALDI Analysis of Untreated and 5FU Treated Colo 205 Cell Lysate for Identification of Differentially Expressed Proteins

Two-dimensional gel electrophoresis was carried out to identify differentially expressed proteins on treatment with anticancer agents specifically, colon/colorectal cancer cell line (NCCS, Pune, India) was treated with 5-FU (Fivoflu, Dabur Pharma Ltd, India). Untreated cells (cells, not treated with 5FU) were taken as control.

100 μM of 5-FU was added to colo 205 cells seeded at 80% confluence and were harvested after 24 hrs of treatment. Cells were lysed in lysis buffer (Urea 0.54 g/ml, CHAPS 40 mg/ml and DTT 20 mg/ml) and the clear supernatant of treated and untreated (not treated with 5-FU) cell lysate were subjected to isoelectric focusing (Bio-Rad, Protean IEF Cell) on separate 17 cm IPG strips of 4-7 pH range.

Second dimension electrophoresis was subsequently carried out on a 12% SDS-PAGE (Protean II XL electrophoresis system, Bio-Rad). The gel was silver stained for visualization of the protein spots and a digitized image of the 2D gel was generated, using GS 800 Densitometer (Bio-Rad). The analysis of the spots of 5-FU treated as well as untreated cells were carried out using PDQuest gel analysis software Ver 7.2 (Bio-Rad).

The protein spots showing more than 2-fold change in intensity in 5-FU treated and untreated samples were further analyzed. The in-gel digestion of the excised gel pieces was performed using a sequencing grade trypsin (Sigma, USA). Peptides were then extracted by adding 100 μl of 60% (v/v) acetonitrile containing 5% (v/v) tri-fluoroacetic acid and dried on a speed vac. The dried samples were reconstituted in 10 μl of 0.1% (v/v of 50% acetonitrile) trifluoroacetic acid and peptides were detected on a Matrix-Assisted Laser Desorption/Ionisation-Time of Flight Mass Spectrometry (MALDI-TOF MS) (Voyager-DE STR). The peptide list was searched through the mammalian subset of the theoretical peptide databases of National Centre for Biotechnology Information (NCBI), SWISS-PROT, Mascot and Profound. Known keratin masses and trypsin auto digest products were excluded from the searches.

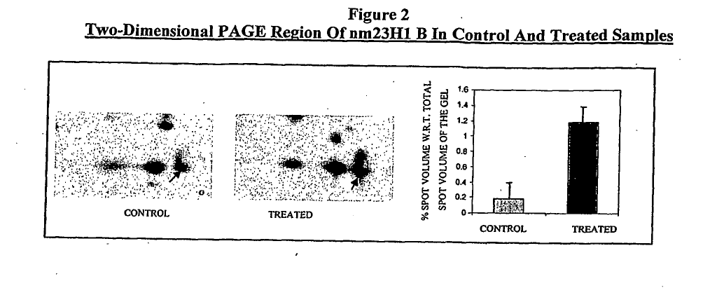

It was found that nm23H1 was more than 6-fold up regulated in 5-FU treated cells as compared to untreated one (FIG. 2). On a closer inspection, we found that it was the 19.6 kDa form of the protein, which is coded by nm23H1-B gene.

### Example 2

### Determination of Differential Expression of nm23H1-A and nm23H1-B at RNA Level in 5-FU Treated Colon Cancer Cell Lines (Colo 205, HT29)

The over expression of nm23H1-B was checked at RNA level by RT-PCR. Colo 205 cells were seeded in 6 well plates at 80% confluence and treated with 100 μM of 5FU for 24 hrs. Subsequently, 1 ml of Trizol reagent (Invitrogen) was added to the cells after a phosphate buffer saline washing for extraction of RNA. The total RNA was extracted using chloroform-isopropyl alcohol method. Finally, the pellet was washed with 70% ethanol and dried on a speed-vac. Control RNA from untreated cells was also extracted in a similar fashion (cells, not treated with 5FU.

RT-PCR was carried out using one step RT-PCR Kit (Qiagen) according to manufacturer's instructions. 1 μg of RNA was taken and mixed with specific primers of nm23H1A & nm23H1B respectively (Table 1, SEQ ID: 3,4,5). β₂-micro globulin (primer sequence, Table 1, SEQ ID: 6,7) was used as a control. The PCR amplified samples were then analyzed on a 1.5% agarose gel.

On analyzing the samples, it was found that there was an up regulation in the nm23H1-B gene whereas, nm23H1-A showed insignificant change in the expression level. β₂-micro globulin was used as an internal control.

TABLE 1
List of primers and probes used in the
study of nm23H1-A and nm23H1-B expression
and cloning experiments

| S.No | SEQ ID | Sequence |
|---|---|---|
| 1 | SEQ ID 3 | 5′GTT CAA ACC TAA GCA GCT GGA AAA3′ |
| 2 | SEQ ID 4 | 5′CAG GAG TAA ATC AGC CTG GTG TGC |
| | | AG3′ |
| 3 | SEQ ID 5 | 5′CAA GCC GAT CTC CTT CTC TG R |
| 4 | SEQ ID 6 | 5′ACC CCC ACT GAA AAA GAT GA |
| 5 | SEQ ID 7 | 5′ATC TTCA AAC CTC CAT GAT G |
| 6 | SEQ ID 8 | 5′CATGCAAGCTTCCGAAGATCT |
| 7 | SEQ ID 9 | 5′GGCCCTGAGTGCATGTATTTC |
| 8 | SEQ ID 10 | 5′ACACTACGTTGACCTGAA |
| 9 | SEQ ID 11 | 5′GAT GGG GAA TTC AG ATG GTG CTA |
| | | CTG TCT ACT TT |
| 10 | SEQ ID 12 | 5′AGC TGG GAA TTC CC ATG GCC AAC |
| | | TGT GAG CGT AC |
| 11 | SEQ ID 13 | 5′TCT GCC GGA TCC CC TTC ATA GAT |
| | | CCA GTT CT3′ |

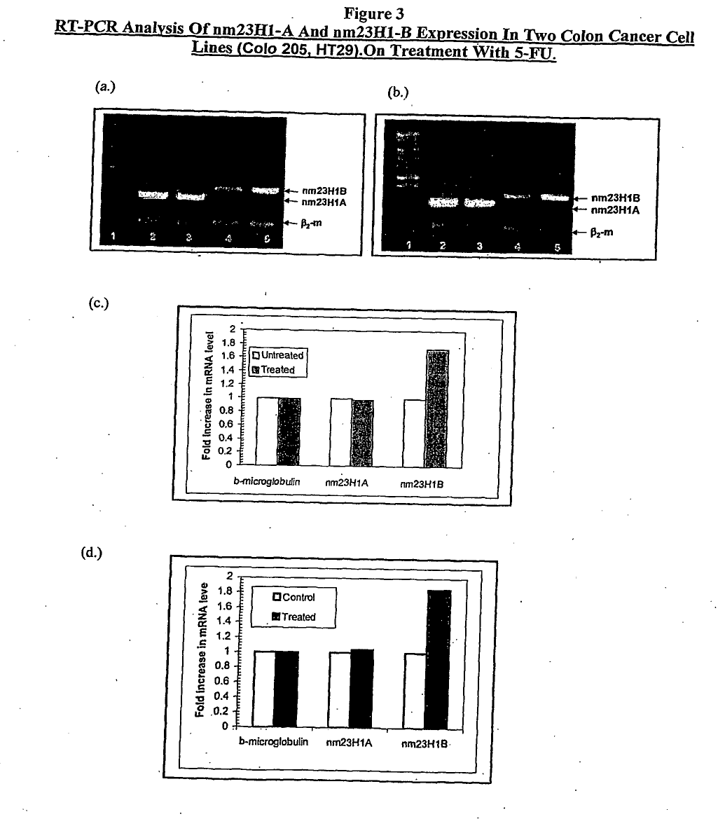

Studies using RT-PCR and real time PCR suggested that it was the nm23H1-B gene which was getting up regulated, whereas, there was an insignificant change in the expression levels of nm23H1-A (FIGS. 3a & 3c). Furthermore, to confirm that the up regulation of nm23H1-B gene is cell line dependent or not, we have carried out similar experiments on another colon cancer cell line, HT29 and got similar results confirming that the up-regulation of nm23H1B gene is not specific to a colon cancer cell line (FIGS. 3b & 3d).

### Example 3

### Time and Dose Dependent Expression of nm23H1-B Gene in 5FU Treated Colon Cancer Cells

To study the effect of drug concentration and duration of treatment on expression of nm23H1 gene, HT29 cells were treated with different concentrations of 5FU (3.1 μM, 6.2 μM, 12.5 μM, 25 μM, 50 μM, 100 μM, 250 μM) for 24 hours in the first set and in the second set HT29 cells were treated with 100 μM of 5FU for different durations (3 h, 6 h, 9 h, 15 h, 24 hrs). RNA was isolated as described in Example 2 and PCR amplified using real time PCR (ABI Prism 7700™ sequence detector system, Applied Biosystems). The oligonucleotide primers (Table 1, SEQ ID: 8,10) and hybridization probes (Table 1, SEQ ID: 9) for PCR targeted for amplification of nm23H1 gene (both nm23H1-A & nm23H1-B) were used. Data was adjusted by measuring intracellular GAPDH concentration with real time detection RT-PCR according to the manufacturer's instructions.

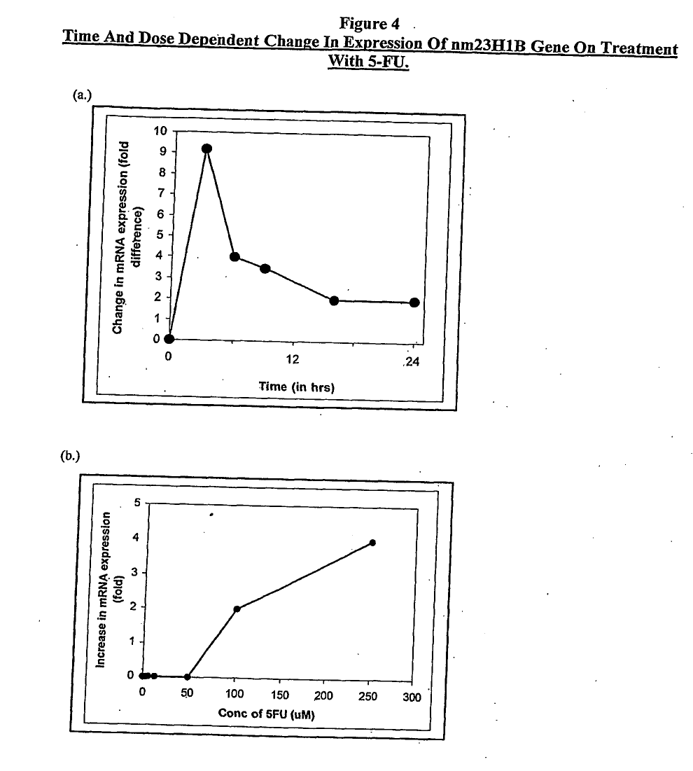

It was found that there was a time (FIG. 4a) and dose (FIG. 4b) dependent change in the expression of nm23H1-B gene confirming the validity of the target.

### Example 4

### Localization of nm23H1-A & nm23H1-B Gene in 5FU Treated Colon Cancer Cells

Intracellular localization of both nm23H1-A & nm23H1-B gene was evaluated by Western Blot analysis. Colo 205 cells were treated with 100 μM 5FU for 24 hours and cells were removed from their dishes by gentle scraping. Cells were washed with cold PBS and then resuspended in cold nuclei extraction buffer (320 mM sucrose, 5 mM Mgcl₂, 10 mM HEPES, 1% Triton X-100 at pH 7.4) at a ratio of 1 ml per million cells. Nuclear fraction was then pelleted by centrifugation at 2000×g and washed twice with nuclei washing buffer (same as nuclei extraction buffer without Triton X-100). Supernatant was collected separately as a cytosolic fraction. Protein was estimated in both nuclear and cytosolic fraction and equal amount of protein was loaded on a 15% SDS PAGE followed by electrobloting on to a polyvinylidene difluoride membrane. Transferred proteins were incubated with blocking buffer containing 3% bovine serum albumin in phosphate-buffered saline. Polyclonal antibody specific for nm23 protein (Santa Cruz) and peroxidase-labeled goat anti-rabbit IgG (Sigma) were used to probe the target gene.

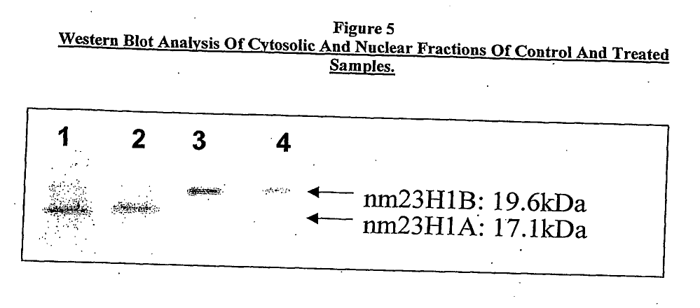

It was observed that the nm23H1-B protein was localized in the cytosolic fraction whereas; nm23H1-A was located in the nuclear fraction of the cells (FIG. 5). Further, as expected there was also an up-regulation of nm23H1-B protein only. Interestingly, a report by Niitsu et al have shown that cytoplasmic nm23 expression in lymphoma cells correlated significantly with the serum nm23H1 levels and also had a significant correlation between patients with cytoplasmic nm23-positive lymphoma and those with performance status 2-4, stage III/IV, bulky mass, B symptoms, elevated serum level of soluble interleukin 2 receptor, and elevated serum level of C-reactive protein (22). It looks very tempting to speculate that possibly it is the nm23H1-B protein, which is the true marker and not nm23H1-A. A good and sensitive immunoassay thus can be developed for screening using the patient sera samples.

### Example 5

### Differential mRNA, Expression Study of nm23H1-A and nm23H1-B in Colon Cancer Cell Lines Treated with Different Anticancer Agents

To study the effect of other anticancer agents on expression of nm23H1-B gene, we took representative of different classes of anticancer agents like alkylating agents (cisplatin), topoisomerase I and II inhibitors (CPT-11), RNA/DNA antimetabolites (5-FU) and antimitotic agents (paclitaxel).

Cells were treated with different anticancer agents for 24 hrs and RNA was isolated. Subsequently, both RT-PCR and real time PCR was carried out as described in Example 2.

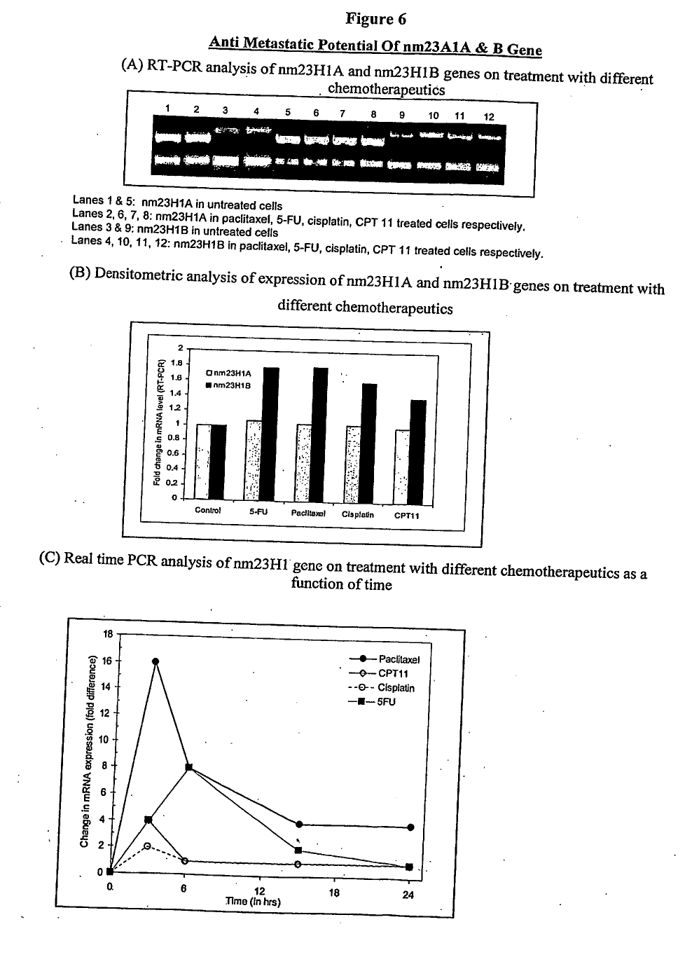

It was found that the there was a similar trend with up-regulation of the expression of the nm23H1-B gene on treatment with anticancer agents (FIG. 6), although the level of up regulation varied among the drugs.

### Example 6

### Evaluation of Anti Metastatic Potential of nm23H1-A and nm23H1-B in Metastatic, Murine Melanoma B16F10 Cells

Anti metastatic potential of nm23H1-A & nm23H1-B Gene was Checked in metastatic, murine melanoma, B16F10 cells. The full-length gene of nm23H1-A & nm23H1-B was cloned in EGFP vector (Clontech) to get GFP fusion proteins. Specific primers for nm23H1-A (Table 1, SEQ ID: 11,13) and nm23H1-B (Table 1, SEQ ID: 12,13) were used to amplify the genes selectively from cDNA of HT29 colon cancer cells. Both vector and inserts were digested with restriction enzymes (EcoR1/BamH1, NEB) and ligated using quick ligase kit (Qiagen). The ligated construct was transformed in E coli cells and positive clones of nm23H1-A & nm23H1-B were screened through restriction digestion and DNA sequencing. Midi prep was carried out to get sufficient plasmid DNA of EGFP, EGFP-nm23H1-A and EGFP-nm23H1-B for further studies.

Transfection was carried out in B16F10 cells to evaluate the metastatic potential of these genes by chemotaxis assay. B16F10 cells were plated in six well plates at 90% confluency in DMEM media without antibiotics. 4 μg of plasmid DNA of each construct was diluted in 250 uL of DMEM media without serum and antibiotics. 10 ul of Lipofectamine™ 2000 reagent (Invitrogen) was also diluted in a similar way. After 5 minutes of incubation at room temperature diluted DNA and lipofectamine were mixed together to form a complex. After 20 minutes of incubation at room temperature the complex was added to the cells in 2 ml of DMEM media without sera and antibiotics. Plate was incubated inside the CO₂ incubator for 6 hours and then media was replaced with complete DMEM media. GFP expression was checked after 24 hours under fluorescence microscope. These transfected cells were starved overnight in DMEM media containing 1% BSA. Subsequently, the cells were collected by trypsinization and cell pellet was washed with DMEM media containing 1% BSA. Cells were then counted in haemocytometer and 0.2 million cells were taken for chemotaxis assay.

Chemotaxis assay was done in a 24 well plate using 8.0 μM polycarbonate membrane (10 mM) tissue culture inserts (Nunc). These inserts were placed in 24 well plates and 0.2 million of transfected cells were added on top of the membranes. 600 ul of NIH3T3 soup was added in the lower chamber as a chemoattractant. Plate was incubated overnight in the CO₂ incubator. Subsequently, the migrated cells were counted under microscope.

Although the transfection of B16F10 metastatic, murine melanoma cell line with nm23H1-A and nm23H1-B showed that the migration efficacy of this cell line was significantly compromised in a chemotaxis assay by both the isoforms of nm23H1, but it was the nm23H1-B transfected cells which showed an increased reduction in the number of cells migrating (by approximately two fold) over nm23H1-A-transfected cells. Therefore, it looks very plausible to use nm23H1-B gene as a candidate for gene therapy for cancer.

1. A marker for diagnosis and/or prognosis of cancer in mammals comprising nm23H1-B gene.

2. A method for diagnosis and/or prognosis of cancer which comprises treating subject cells with one or more chemotherapeutic agents and identifying the alteration in the expression of nm23H1-B gene or protein or both for detecting the indicating the presence of cancer.

3. The method as claimed in claim 2, wherein said method is in vivo.

4. The method as claimed in claim 2 or wherein said one or more chemotherapeutic agents are selected from the group consisting of 5 FU, CPT 11 and cisplatin.

5. (canceled)

6. The method as claimed in claim 2 wherein said cancer is cancer of colon, colorectal, breast, prostate, renal, lung or pancreas.

7. (canceled)

8. The use of the nm23H1B gene as a therapeutic agent in gene therapy.

9. The use of nm23H1B gene as claimed in claim 8 in combination with any other therapy.

10. The method as claimed in claim 3, wherein said one or more chemotherapeutic agents are selected from the group consisting of 5 FU, CPT 11 and cisplatin.

11. The method as claimed in claim 3, wherein said cancer is cancer of colon, colorectal, breast, prostate, renal, lung or pancreas.

12. The method as claimed in claim 4, wherein said cancer is cancer of colon, colorectal, breast, prostate, renal, lung or pancreas.

13. The method as claimed in claim 10, wherein said cancer is cancer of colon, colorectal, breast, prostate, renal, lung or pancreas.

14. A method for treating cancer comprising administering nm23HIB gene to a patient in need thereof.

15. The method according to claim 14, further comprising another therapy for treating cancer.


SEQUENCE LISTING (13 sequences)

SEQ ID NO: 1 - DNA, 987 bp, Human
ggtgcaagtgctgcgaaccacgtgggtcccgggcgcgtttcgggtgctggcggctgcagtcggagttcaaacctaagcagctggaagggccctgtggctaggtaccatagagtctctacacaggagtaaatcagcctggtgtgcaggggaggcagacacacaaacagacaattggactacagtgctaagatgctgtaagaagaggttaactaaaggacaggaagatggggccaagagatggtgctactgtctactttagggatcgtctttcaaggcgaggggcctcctatctcaagctgtgatacaggaaccatggccaactgtgagcgtaccttcattgcgatcaaaccagatggggtccagcggggtcttgtgggagagattatcaagcgttttgagcagaaaggattccgccttgttggtctgaaattcatgcaagcttccgaagatcttctcaaggaacactacgttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttcccatgggcagaggaccaggctgtaggaaatctagttatttacaggaacttcatcataatttggagggaagctcttggagctgtgagttctccctgtacagtgttaccatccccgaccatctgattaaaatgcttcctcccagcataggattcattgagttggttacttca

SEQ ID NO: 2 - protein, 177 aa, Human
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 3 - DNA, 24 bp, Artificial Sequence
  Primer
gttcaaacctaagcagctggaaaa

SEQ ID NO: 4 - DNA, 26 bp, Artificial Sequence
  Primer
caggagtaaatcagcctggtgtgcag

SEQ ID NO: 5 - DNA, 21 bp, Artificial Sequence
  Primer
caagccgatctccttctctgr

SEQ ID NO: 6 - DNA, 20 bp, Artificial Sequence
  Primer
acccccactgaaaaagatga

SEQ ID NO: 7 - DNA, 20 bp, Artificial Sequence
  Primer
atcttcaaacctccatgatg

SEQ ID NO: 8 - DNA, 21 bp, Artificial Sequence
  Primer
catgcaagcttccgaagatct

SEQ ID NO: 9 - DNA, 21 bp, Artificial Sequence
  Primer
ggccctgagtgcatgtatttc

SEQ ID NO: 10 - DNA, 18 bp, Artificial Sequence
  Primer
acactacgttgacctgaa

SEQ ID NO: 11 - DNA, 34 bp, Artificial Sequence
  Primer
gatggggaattcagatggtgctactgtctacttt

SEQ ID NO: 12 - DNA, 34 bp, Artificial Sequence
  Primer
agctgggaattcccatggccaactgtgagcgtac

SEQ ID NO: 13 - DNA, 31 bp, Artificial Sequence
  Primer
tctgccggatccccttcatagatccagttct